(19) United States
(12) Patent Application Publication
(10) Pub. No.: US 2023/0032796 A1
(43) Pub. Date: Feb. 2, 2023

(54) Personal Lubricant Composition

(71) Applicant: Reckitt Benckiser Health Limited, Slough (GB)

(72) Inventors: Thomas Carvell, Hull (GB); Philip Towell, Bangkok (TH)

(21) Appl. No.: 17/785,959

(22) PCT Filed: Dec. 17, 2020
(86) PCT No.: PCT/GB2020/053256

§ 371 Date: Jun. 16, 2022

(30) Foreign Application Priority Data
Dec. 17, 2019 (GB) 1918617.0

(51) Int. Cl. A61K 8/73; A61K 8/34; A61K 9/00

(52) U.S. Cl. CPC A61K 8/731; A61K 8/345; A61K 9/0034

(57) ABSTRACT

The present invention provides a personal lubricant composition comprising a humectant and a thixotropic agent, wherein the thixotropic agent comprises a combination of: i) one or more celluloses; and ii) one or more alkali metal carboxyalkylcelluloses. The composition may be sprayable and provided in a pump spray device or squeeze-type spray bottle. The invention further provides a method of providing personal lubrication, comprising dispensing the composition to a target area, as well as the use of the composition to provide lubrication during sexual activity.

The present invention is directed to a personal lubricant composition. In a preferred embodiment, the present invention is directed to a sprayable personal lubricant that is of a lower osmolality than existing sprayable personal lubricants, while maintaining lubricity as well as good texture, skin-feel and other desirable properties.

Personal lubrications for intimate contact are well known. Typically, personal lubricants are marketed as liquids, jellies, gels or suppositories. Examples of such products include K-Y® Jelly, Astroglide®, K-Y® Liquid, K-Y® Ultragel™. More recently, K-Y® Warming Liquid was introduced to the marketplace. K-Y® Warming Liquid is a water soluble, anhydrous composition that warms on contact with the human body, while providing lubrication.

Most of the commercially available personal lubricant products are used by first applying to the hand or fingers of the user and then to the intimate area. This can be undesirable and messy. Additionally, some individuals are averse to applying a personal lubricant directly to the genital regions.

One method to deliver a lubricant hands-free is through spraying, for example by dispensing the lubricant from an atomizer or a pressurized container. However, not all lubricants are capable of being sprayed. Personal lubricants, in particular, typically have a high viscosity that prevents them from being sprayed. Lowering the viscosity of such personal lubricant compositions by dilution in liquid facilitates spraying, but causes a loss of lubricity.

US 2007/0059250 and US 2007/059266 describe personal lubricant compositions that are capable of being sprayed to a target area while maintaining lubricity. However, the compositions in the specific examples of these documents rely on high levels of humectant (≥30 wt %), specifically glycerine and/or propylene glycol, to provide the desired sprayability and lubricity. This results in the compositions having an osmolality that is higher than the World Health Organisation recommended level of below 1200 mOsm/kg (see “Use and procurement of additional lubricants for male and female condoms: WHO/UNFPA/FH1360 Advisory Note. WHO Advisory Note. 2012:1-8.”).

Moreover, it has been found that the compositions described in US 2007/0059250 and US 2007/059266 tend to spray as a jet or a series of large polydisperse droplets from a typical atomiser or pressurized container, rather than a gentle, mess-free mist.

WO2018/200014 discloses a method of contraception using a thixotropic Bingham plastic fluid, intended to be applied inside the vaginal vault. It may comprise a mixture of microcrystalline cellulose and sodium carboxymethylcellulose, but does not include other elements such as propylene glycol or glycerine. Embodiments of the composition are said to be additionally suitable for use as a personal lubricant. However, the actual properties of the compositions of this document leave much to be desired for the consumer, such as in texture, skin feel and “stickiness”, and levels of lubricity, when used as a lubricant for sexual activity rather than for contraception.

It would be desirable to provide a personal lubricant composition that is simultaneously sprayable and lubricious while having a low osmolality and desirable sensorial properties.

It would alternatively or additionally be desirable to provide a personal lubricant composition that can be dispensed as a fine mist.

According to a first aspect of the present invention, there is provided a personal lubricant composition comprising a humectant and a thixotropic agent, wherein the thixotropic agent comprises a combination of: i) one or more celluloses; and ii) one or more alkali metal carboxyalkylcelluloses. As used herein, “cellulose” does not include cellulose ethers or cellulose esters, but the one or more celluloses may have any molecular weight, and may be microcrystalline cellulose.

The present inventors have found that the inclusion of this specific thixotropic agent may enable the composition to be sprayed as a gentle, fine mist without requiring the high levels of humectant used in the compositions exemplified in US 2007/0059250 and US 2007/059266. Surprisingly, the lubricity of the composition may be maintained even with low levels of humectant. Advantageously in an embodiment, low levels of humectant can be used which result in a low-osmolality composition. Accordingly, described herein are personal lubricant compositions that are sprayable while exhibiting low osmolality and good lubricity. The compositions may be non-sticky and leave no perceivable residue.

It has also surprisingly been found that the specific thixotropic agent disclosed herein may result in a composition that provides consistent lubricity over a wide range of shear rates, both before and after spraying. This means that the personal lubricant composition can be designed to give good lubricity regardless of the speed of the sexual activity, even after spraying.

The present invention will now be described further. In the following passages different aspects/embodiments of the invention are defined in more detail. Each aspect/embodiment so defined may be combined with any other aspect/embodiment or aspects/embodiments unless clearly indicated to the contrary. In particular, any feature indicated as being preferred or advantageous may be combined with any other feature or features indicated as being preferred or advantageous.

As used herein, the phrase “personal lubricant composition” means those types of compositions that supply lubrication during personal or sexual relations/activity, preferably during sexual activity. The personal lubricant composition of the present invention may be applied to the vagina, vaginal area, perineum, anal area, penis, or oral cavity. Preferably, it is suitable for lubrication of at least the vagina, vaginal area, perineum, anal area, or penis, preferably for lubrication of at least the vagina, vaginal area or penis. The composition may also be applied to devices such as medical devices, gloves, or sexually-related devices such as vibrators, sexual aids, and the like. For example, it may be used for lubrication of medical devices or gloves employed during gynaecological examinations.

The personal lubricant composition as disclosed herein is preferably sprayable. By “sprayable” it is meant that the composition can be expressed as a mist of particles from a pump spray device or squeeze-type spray bottle. The term “mist” is used herein to refer to a spray pattern that does not have clearly defined outer boundaries.

In one embodiment, the pump spray device comprises a spray pump actuator combination.

In one embodiment, the pump spray device comprises a swirl chamber. In this embodiment, the composition is typically drawn up into the chamber by a dip tube. Once inside the swirl chamber, the composition breaks up into minute particles or to a fine spray, which is then expelled.

Once the spray pump has been primed, the composition is delivered to the defined target area in predetermined amounts. In one embodiment, the pump may require at least two strokes to prime. In another embodiment, the pump may require at least three strokes to prime. During priming, the composition is drawn up the dip tube, into the swirl chamber, through the actuator and eventually expelled. In one embodiment, the spray pump delivers 120 to 160 μL of composition per a single stroke into the defined target area.

In order to be sprayable in a preferred embodiment, the compositions of this invention should be capable of being atomized into particles having a size distribution as follows, given for the 10th, 50th and 90th percentiles:

| | Particle Size (μm) | | |
|---|---|---|---|
| Distance | D[v, 0.1] | D[v, 0.5] | D[v, 0.9] |
| 25 cm | 60 | 100 | 200 |

Preferably, particle size at 25 cm should be at most about 60 μm in the 10th percentile, at most about 100 μm in the 50th percentile and at most about 200 μm in the 90th percentile. Preferably, the particle size is measured using a Malvern Spraytec Laser Diffraction instrument.

Preferably, the composition disclosed herein is sprayable from a pump spray device or squeeze-type spray bottle, more preferably such that the composition upon spraying is atomized into particles having a size distribution defined by a D[v, 0.1] of at most about 60 μm, a D[v, 0.5] of at most about 100 μm and a D[v, 0.9] of at most about 200 μm. The particle size distribution is preferably determined at a distance of 25 cm from the source of the spray and/or using a Malvern Spraytec Laser Diffraction instrument.

In one embodiment, the spray pump may be a two-way pump, that is, the composition contained within the pump can be dispensed from an upright or inverted configuration such that the user can spray “upside down”.

Preferably, the compositions and methods of this invention enable the compositions to be dispensed from a location remote from the target area with minimal dispersion to undesired areas. For example, the compositions may be sprayed at a target area with reasonable accuracy from a distance of at least about 8 cm, preferably at least about 30 cm.

The composition of the present invention includes a thixotropic agent, which comprises: i) one more celluloses; and ii) one or more alkali metal carboxyalkylcelluloses. In an embodiment, the thixotropic agent consists of one cellulose and one alkali metal carboxyalkylcellulose. The inclusion of the thixotropic agent means that the composition is preferably a non-Newtonian thixotropic fluid, exhibiting a reduced apparent viscosity while being subjected to shear forces, but a high apparent viscosity while at rest; this property permits application by spraying with readily available pump spray devices or squeeze-type spray bottles immediately following the application of a shearing force (such as those created by vigorously shaking the product container), but causes the sprayed material to remain at least temporarily relatively immobile on mucosal membranes or the skin. As explained above, the present inventors have found that the inclusion of the particular thixotropic agent described herein, specifically a combination of one or more celluloses and one or more alkali metal carboxyalkylcelluloses, enables the composition to be sprayed as a gentle, fine mist and remain lubricious, without requiring the high levels of humectant (e.g. glycerine and/or propylene glycol) used in the compositions of the prior art. Preferably, the alkyl group of the one or more alkali metal carboxyalkylcelluloses is selected from methyl, ethyl, propyl and butyl, more preferably methyl or ethyl, most preferably methyl. Preferably, the alkali metal of the one or more alkali metal carboxyalkylcelluloses is selected from lithium, sodium and potassium, more preferably sodium.

As set out above, it has also surprisingly been found that the specific thixotropic agent disclosed herein ideally results in a composition that provides consistent lubricity over a wide range of shear rates, both before and after spraying. This means that the personal lubricant composition delivers on its purpose regardless of the speed of the sexual activity, even after spraying.

Preferably, the composition has a very rapid rate of viscosity recovery, following withdrawal of the shearing force.

Preferably, the thixotropic agent comprises a combination of microcrystalline cellulose and an alkali metal carboxyalkylcellulose as described above, more preferably sodium carboxymethylcellulose. In an embodiment, the thixotropic agent comprises or consists of 85-95 wt % (preferably 86-92 wt %) of the one or more celluloses and 5-15 wt % (preferably 8-14 wt %) of the one or more alkali metal carboxyalkylcelluloses. Typically the thixotropic agent comprises or consists of 85-95 wt % microcrystalline cellulose and 5-15 wt % sodium carboxymethylcellulose. More typically the thixotropic agent comprises or consists of 86-92 wt % microcrystalline cellulose and 8-14 wt % sodium carboxymethylcellulose. The present inventors have found that such a thixotropic agent can provide an excellent balance between sprayability and lubricity, obviating the need to include significant amounts of humectant. Preferably the thixotropic agent consists of the combination of one or more celluloses and one or more alkali metal carboxyalkylcelluloses, or more preferably the combination of microcrystalline cellulose and sodium carboxymethylcellulose.

Preferably, the thixotropic agent is present in the composition in an amount of: i) at least 1 wt %, at least 2 wt %, at least 3 wt %, at least 3.1 wt %, at least 3.2 wt %, at least 3.3 wt %, at least 3.4 wt %, at least 3.5 wt %, at least 3.6 wt %, at least 3.7 wt %, or at least 3.8 wt %; and/or ii) up to 10 wt %, up to 9 wt %, up to 8 wt %, up to 7 wt %, up to 6 wt %, up to 5 wt %, up to 4 wt %, up to 3.9 wt %, up to 3.8 wt %, up to 3.7 wt %, up to 3.6 wt %, up to 3.5 wt %, up to 3.4 wt %, or up to 3.3 wt %. In an embodiment, it is present from 1 to 10 wt % of the composition, more preferably from 2 to 5 wt %, and most preferably from 2 to 4 wt %. An upper limit of 10 wt % avoids producing high viscosities which impede spraying with the usual devices.

The composition of the invention further comprises a humectant. Preferably, the composition comprises less than 25 wt % humectant by weight of the composition, more preferably less than 20 wt % humectant, or up to 15 wt %, up to 10 wt %, or up to 9 wt % humectant. It is to be understood that in these embodiments, where two or more humectants are present, it is the total amount of humectant that is less than the amounts stated. Most preferably, the composition comprises at least 2 wt % humectant, preferably at least 3 wt %, at least 4 wt %, at least 5 wt %, at least 6 wt %, or at least 7 wt % humectant. In an embodiment, it comprises from 5 to 15 wt % humectant, or 7 to 9 wt % humectant. Preferably, the humectant is selected from the group consisting of glycerine, propylene glycol (1,2-propanediol), 1,3-propanediol, polyethylene glycol and combinations of two or more thereof, preferably from the group consisting of glycerine, propylene glycol and a combination thereof.

High levels of humectant, specifically glycerine and/or propylene glycol, are employed in the compositions exemplified in US 2007/0059250 and US 2007/059266 to ensure good sprayability and lubricity. In the present invention, the inclusion of the combination of one or more celluloses and one or more alkali metal carboxyalkylcelluloses allows for good sprayability and lubricity while humectant is still present but the levels of humectant may be reduced. This, in turn, provides opportunity for the provision of a low-osmolality composition, in accordance with the WHO guidelines discussed above.

Preferably, the humectant is a combination of glycerine and propylene glycol (1,2-propanediol), more preferably wherein the glycerine and propylene glycol are present in a weight ratio of from 2:3 to 3:2. The present inventors have found that such ratios of glycerine to propylene glycol provides excellent skin-feel to the lubricant. Most preferably, the glycerine and propylene glycol are present in a weight ratio of about 1:1.

Preferably, the weight ratio of humectant to thixotropic agent is less than 4:1, preferably from 2:1 to 3:1. In these embodiments, the humectant is preferably a combination of glycerine and propylene glycol.

Preferably, the composition has an osmolality of less than 1200 mOsm/kg, preferably from 1000 to 1100 mOsm/kg. Preferably the osmolality is determined using the method described in United States Pharmacopeia (USP) reference standard 785, specifically USP43-NF38, the contents of which are incorporated herein by reference.

Preferably, the composition has a viscosity of at least 800 mPa·s at a temperature of 25° C. and a shear rate of 10 s⁻¹ more preferably from 1000 to 1500 mPa·s. Preferably, the composition has a viscosity of from 100 to 600 mPa·s at a temperature of 25° C. and a shear rate of 1000 s⁻¹, more preferably from 200 to 500 m Pa·s. It is to be understood that these values refer to the viscosity of the composition prior to spraying.

Preferably, the composition has a viscosity after spraying of at least 400 mPa·s at a temperature of 25° C. and a shear rate of 10 s⁻¹, more preferably from 500 to 800 mPa·s. Preferably, the composition has a viscosity after spraying of from 100 to 600 mPa·s at a temperature of 25° C. and a shear rate of 1000 s⁻¹, more preferably from 200 to 500 mPa·s. Preferably, the spraying is performed under the conditions described herein.

It has been found that the inclusion of a combination of one or more celluloses and one or more alkali metal carboxyalkylcelluloses as a thixotropic agent can provide a composition whose viscosity as measured at low shear rates (e.g. 10 s⁻¹) is lower after spraying than before, but whose viscosity as measured at higher shear rates (e.g. 1000 s⁻¹) is maintained after spraying. Thus, in some embodiments the composition disclosed herein has a viscosity after spraying of at most 80%, preferably at most 60% of a viscosity before spraying, the viscosity after spraying and the viscosity before spraying being measured at a temperature of 25° C. and a shear rate of 10 s⁻¹. Moreover, in some embodiments the composition disclosed herein has a viscosity after spraying of at least 90%, preferably from 90 to 100% of a viscosity before spraying, the viscosity after spraying and the viscosity before spraying being measured at a temperature of 25° C. and a shear rate of 1000 s⁻¹. Preferably, the spraying is performed under the conditions described herein.

Preferably, the viscosity values described herein are measured using a Discovery Series Hybrid rheometer (DHR2, TA Instruments) fitted with a 40 mm 1° cone measuring system.

Advantageously, the composition of the present invention may have a relatively high resting viscosity (i.e. before and after spraying) meaning that the composition forms a non-drip, mess-free layer that does not migrate or spread once applied to the target area. The composition is sprayable, despite its high resting viscosity, because the thixotropic agent causes the composition to exhibit a significantly reduced apparent viscosity while being subjected to shear forces. In other words, the composition has a low viscosity under high shear conditions.

Lubricity may be quantified in relation to a coefficient of friction value determined from tribology measurements. The coefficient of friction is inversely proportional to the lubricity of a product and is known as “relative lubricity”. Relative lubricity can be calculated from the coefficient of friction data by dividing the numeral one by the corresponding coefficient of friction value. Preferably, the composition of the present invention provides a coefficient of friction of at least 0.05 at a sliding speed of 10000 μm/s as determined by the tribology test method described in the Examples, more preferably from 0.06 to 0.15. It is to be understood that these values refer to the lubricity of the composition prior to spraying. Preferably, the composition after spraying provides a coefficient of friction of from 0.05 to 0.15 at a sliding speed of 10000 μm/s as determined by the tribology test method described in the Examples, more preferably from 0.06 to 0.15. Preferably, the coefficient of friction after spraying is within 20%, more preferably within 10%, of the coefficient of friction before spraying. Thus, the composition of the present invention retains a high lubricity even after being sprayed. Preferably, the spraying is performed under the conditions described herein.

Preferably, the composition is aqueous. Preferably, the composition comprises at least 60 wt % water by weight of the composition, preferably at least 65 wt % water, more preferably from 70 to 95 wt % water.

Preferably, the composition has a pH of from 3.0 to 5.0, preferably from 3.5 to 4.5, preferably from 4.0 to 4.3, preferably about 4.2. This pH is suitable for the vaginal area. Moreover, the present inventors have found that in embodiments wherein the thixotropic agent is a combination of microcrystalline cellulose and sodium carboxymethylcellulose, the resting viscosity of the composition at a given concentration of thixotropic agent is at a maximum when the pH is within this range. The composition may comprise a pH adjuster, such as sodium hydroxide, to bring the pH to within the desired range.

In some embodiments, the composition comprises a buffer. Suitable buffers for use in the present invention include, for example, lactic acid/sodium lactate. Preferably, however, the composition does not contain a buffer. This means that there will be less altering effect on skin pH.

Preferably, the composition comprises a preservative, preferably selected from the group consisting of benzoic acid, parabens, potassium sorbate, sodium benzoate, phenoxyethanol and combinations of two or more thereof. Preferably, the preservative is present in an amount of from 0.05 to 5 wt % by weight of the composition, more preferably from 0.1 to 1 wt %.

Preferably, the composition does not comprise a pharmaceutically active ingredient. Preferably, the composition does not comprise a non-steroidal anti-inflammatory drug (NSAID), for example, flurbiprofen, ketoprofen or diclofenac, or a pharmaceutically acceptable salt thereof. Preferably, the composition does not comprise a pharmaceutically active ingredient selected from hexylresorcinol, benzocaine, dextromethorphan, menthol lidocaine, amyl metacresol and 2,4-dichlorobenzyl alcohol or pharmaceutically acceptable salts thereof.

In an especially preferred embodiment there is provided a personal lubricant composition comprising from 2 to 5 wt % of a thixotropic agent and from 5 to 15 wt % of a humectant,
wherein the thixotropic agent is a combination of one or more celluloses and one or more alkali metal carboxyalkylcelluloses,
wherein the humectant is a combination of glycerine and propylene glycol,
wherein the glycerine and propylene glycol are present in a weight ratio of from 3:2 to 2:3, and
wherein the composition has an osmolality of less than 1200 mOsm/kg and a pH of from 4.0 to 5.0.

According to a second aspect of the invention, there is provided a pump spray device or squeeze-type spray bottle comprising the personal lubricant composition as described in relation to the first aspect of the invention.

According to a third aspect of the invention, there is provided a method of providing personal lubrication comprising dispensing a personal lubricant composition according to the first aspect of the invention to a target area, preferably from a pump spray device or squeeze-type spray bottle. In an embodiment, the method is a method of providing lubrication during sexual activity. In an embodiment, the target area is on the human body, preferably a genital or anal area of the human body, preferably the vagina, vaginal area or penis. Preferably, the target area does not comprise the cervix. In another embodiment, the method comprises dispensing the composition onto a target area on a product which is then brought into contact with an anal or genital area of the human body, preferably wherein the product is a vibrator, dildo or other sex toy.

According to a fourth aspect, there is provided the use of a personal lubricant composition according to the first aspect of the invention to provide lubrication during sexual activity.

An embodiment of the present invention will now be described in relation to the following non-limiting figures, in which:

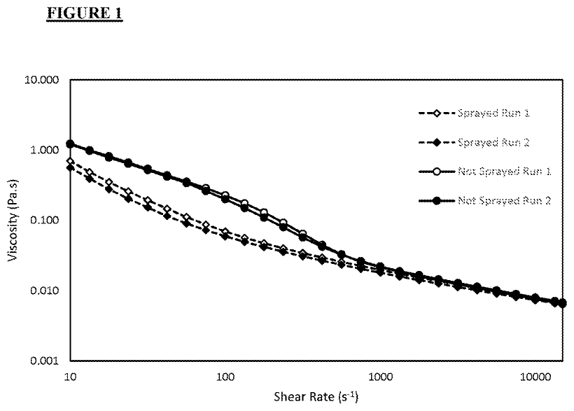

FIG. 1 is a graph showing the measured viscosity of a personal lubricant of the present invention as a function of shear rate in accordance with Examples 1 and 2. The y-axis represents viscosity in Pa·s and the x-axis represents shear rate in s⁻¹. The solid lines represent the viscosity of the lubricant before spraying (the unfilled and filled circles represent first and second measurements respectively). The dashed lines represent the viscosity of the lubricant after spraying (the unfilled and filled diamonds represent first and second measurements respectively).

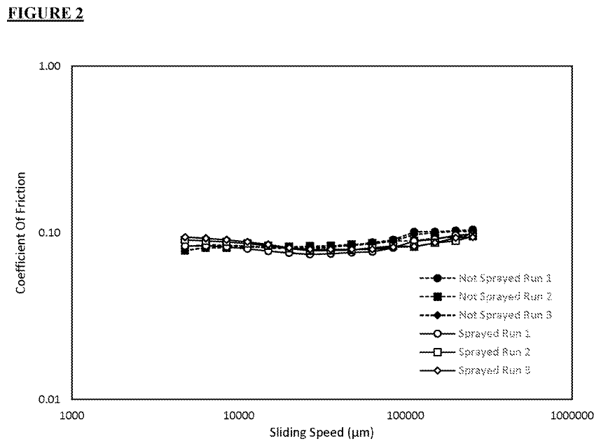

FIG. 2 is a graph showing the measured coefficient of friction of a personal lubricant of the present invention as a function of sliding speed in accordance with Examples 1 and 3. The y-axis represents coefficient of friction and the x-axis represents sliding speed in μm. The dashed lines represent the coefficient of friction of the lubricant before spraying (the filled circles, squares and diamonds represent first, second and third measurements respectively). The solid lines represent the coefficient of friction of the lubricant after spraying (the unfilled circles, squares and diamonds represent first, second and third measurements respectively).

The present invention will now be described in relation to the following non-limiting Examples.

### EXAMPLE 1

A personal lubricant was prepared having the following composition:

| Substance | % w/w |
|---|---|
| Benzoic Acid | 0.10-0.30 |
| Glycerine | 3.5-4.5 |
| Propylene Glycol | 3.5-4.5 |
| DI Water | qs. |
| Microcrystalline cellulose | 3.1-3.3 |
| Sodium | 0.3-0.5 |
| carboxymethylcellulose | |
| NaOH (30%) | 0.01-0.03 |

The composition was prepared by dispersing the thickener in de-ionized water with low shear force. High shear was then applied to activate the thickener (colloidal mill at max rpm. 3-10 min: homogenizer min 150 bar). After activation, the mixture was left to stand for 15 minutes. The propylene glycol, glycerine and perseverative were then dissolved in the liquid phase and mixed with medium stirring. Finally, the NaOH was added and the pH was measured.

The pH was 4.2 and the osmolality was measured as 1045.9 mOsm/kg in accordance with the reference standard 785 of USP43-NF38. The product had a desirable texture and skin-feel, without feeling sticky, giving a very pleasurable sensation during sexual activity.

### EXAMPLE 2

The viscosity of the composition of Example 1 was determined at a temperature of 25° C. before and after spraying on a research rheometer (DHR2, TA Instruments) fitted with a 40 mm 1° cone measuring system. A solvent trap cover was employed to minimise drying of the sample at the exposed edges. Following a 30 s equilibration time at 25° C. the samples were exposed to a 30 s pre-shear at 10 s⁻¹ shear rate sweep, 10 s⁻¹ to 15000 s⁻¹, logarithmically scaled, 8 points per decade of shear rate, shear applied for 30 s at each rate with viscosity calculated over the final 5 seconds of each step.

The results are shown in FIG. 1 and summarised in the following table:

TABLE 1
Viscosity Metrics

| | Viscosity at 10 s⁻¹ | | | Viscosity at 1000 s⁻¹ | | |
|---|---|---|---|---|---|---|
| Sample | Run 1 | Run 2 | Mean | Run 1 | Run 2 | Mean |
| Example 1 (before spraying) | 1.23 | 1.20 | 1.22 | 0.02 | 0.02 | 0.02 |
| Example 1 (after spraying) | 0.70 | 0.56 | 0.63 | 0.02 | 0.02 | 0.02 |

### EXAMPLE 3

The tribology profile of the composition of Example 1 was determined before and after spraying using the same rheometer used in Example 2 (DHR-2, TA Instruments) fitted with a custom 3 balls on plate measuring system. Specifically, the tribology profile of the composition of Example 1 was determined as follows. A tribology assembly was employed that comprised a geometry of 3 glass hemispheres that slid against a lower silicone substrate, under a defined load of IN, onto which the sample had been spread. The rotational angular velocity is ramped from 0.05 rad/s to 20 rad/s, 8 points per decade, each point held for 20 s with the coefficient of friction averaged over the final 15 s. The lower substrate was held at 32° C. throughout. This test was performed in triplicate.

The results are shown in FIG. 2 and summarised in the following table:

| | Coefficient of Friction at 10,000 μm/s | | | |
|---|---|---|---|---|
| Sample | Run 1 | Run 2 | Run 3 | Mean |
| Example 1 (before spraying) | 0.08 | 0.08 | 0.08 | 0.08 |
| Example 1 (after spraying) | 0.08 | 0.09 | 0.09 | 0.09 |

Surprisingly, the inclusion of the thixotropic thickener described herein allows the lubricity of the composition to be retained over a broad range of sliding speeds, both before and after spraying. This cannot simply be explained by viscosity, since the viscosity of the composition has been shown in Example 2 to decrease after spraying when measured at low shear rates.

### EXAMPLE 4

The tribology profile of the composition of Example 1 was compared with two existing commercial products (K-Y Liquid and K-Y Jelly). The coefficient of friction at 10000 μm/s was determined without spraying using the method described in Example 3. The results are shown in the following table:

| Sample | Coefficient of friction (μ) at 10000 μm/s |
|---|---|
| Example 1 | 0.08 |
| K-Y Liquid | 0.08 |
| K-Y Jelly | 0.09 |

The lubricity of the composition of Example 1 is comparable to that of existing commercial personal lubricants, even though it can be sprayed as a fine mist.

The foregoing detailed description has been provided by way of explanation and illustration, and is not intended to limit the scope of the appended claims. Many variations in the presently preferred embodiments illustrated herein will be apparent to one of ordinary skill in the art, and remain within the scope of the appended claims and their equivalents.

1. A personal lubricant composition comprising:
a humectant; and
a thixotropic agent;
wherein the thixotropic agent comprises a combination of:
one or more celluloses; and
one or more alkali metal carboxyalkylcelluloses.

2. The personal lubricant composition according to claim 1, wherein the humectant is propylene glycol, glycerine or a combination thereof.

3. The personal lubricant composition according to claim 1, wherein the composition is sprayable.

4. The personal lubricant composition according to claim 1, wherein the composition has an osmolality of less than 1200 mOsm/kg.

5. The personal lubricant composition according to claim 1, wherein the thixotropic agent comprises a combination of microcrystalline cellulose and sodium carboxymethylcellulose.

6. The personal lubricant composition according to claim 1, wherein the thixotropic agent is present in an amount of from 1 to 10 wt % by weight of the composition.

7. The personal lubricant composition according to claim 1, wherein the humectant is present in an amount of less than 25 wt % by weight of the composition.

8. The personal lubricant composition according to claim 2, wherein the humectant is a combination of glycerine and propylene glycol.

9. The personal lubricant composition according to claim 8, wherein the glycerine and propylene glycol are present in a weight ratio of from 3:2 to 2:3.

10. The personal lubricant composition according to claim 1, wherein the composition has a viscosity of at least 800 mPa·s at a temperature of 25° C. and a shear rate of 10 s⁻¹.

11. The personal lubricant composition according to claim 1, wherein the composition has a pH of from 4.0 to 5.0.

12. A personal lubricant composition comprising:
glycerine and propylene glycol, in a total amount of 5 to 15 wt %; and
one or more celluloses, and one or more alkali metal carboxyalkylcelluloses, in a total amount of 2 to 5 wt %;
wherein the glycerine and propylene glycol are present in a weight ratio of 3:2 to 2:3; and
wherein the composition has an osmolality of less than 1200 mOsm/kg and a pH of from 4.0 to 5.0.

13. An apparatus comprising the personal lubricant composition according to claim 1;
wherein the apparatus is selected from the group consisting of a pump spray device and squeeze-type spray bottle.

14. A method of providing personal lubrication comprising:
dispensing a personal lubricant composition according to claim 1 to a target area.

15. Use of a personal lubricant composition according to claim 1 to provide lubrication during sexual activity.

16. The method of providing personal lubrication according to claim 14, wherein the dispensing is from an apparatus selected from the group consisting of a pump spray device and squeeze-type spray bottle.
